(19) United States
(12) Patent Application Publication
(10) Pub. No.: US 2025/0242125 A1
(43) Pub. Date: Jul. 31, 2025

(54) MULTI-FUNCTIONAL BREATHING TUBE FOR HUMIDIFICATION WITHOUT WATER

(71) Applicant: INNOVATION RESEARCHINSTITUTE OF ZHEJIANGUNIVERSITY OF TECHNOLOGY,SHENGZHOU, Shaoxing (CN)

(72) Inventors: Shuiqing ZHOU, Shaoxing (CN); Weiya JIN, Shaoxing (CN); Qingyu WANG, Shaoxing (CN); Xing HE, Shaoxing (CN); Jian FANG, Shaoxing (CN)

(73) Assignee: INNOVATION RESEARCHINSTITUTE OF ZHEJIANGUNIVERSITY OF TECHNOLOGY,SHENGZHOU, Shaoxing (CN)

(21) Appl. No.: 18/746,406

(22) Filed: Jun. 18, 2024

Related U.S. Application Data
(63) Continuation of application No. PCT/CN2024/092160, filed on May 10, 2024.

(30) Foreign Application Priority Data
Jan. 25, 2024 (CN) 202410106574.0

(51) Int. Cl. A61M 16/10; A61M 11/00; A61M 16/00; A61M 16/06; B05B 17/00

(52) U.S. Cl. CPC A61M 16/109; A61M 11/00; A61M 16/0006; A61M 16/06; B05B 17/0638; A61M 2202/0007; A61M 2202/0208; A61M 2205/127; A61M 2205/36; A61M 2205/8206

(57) ABSTRACT

The invention discloses a multi-functional breathing tube for humidification without water. It includes a breathing mask, the breathing mask is connected with a sputum ejector, the sputum ejector is connected with an anhydrous humidifier atomizer, and the anhydrous humidifier atomizer is connected with a medicated bin. The medicated bin is connected with a breathing hose, the breathing hose is connected with an air source device, and the gas output by the air source device is oxygen. The sputum ejector is connected with the anhydrous humidifier atomizer. The pendulum ball in the sputum ejector resonates with the sputum during the patient's deep exhalation to assist the sputum excretion from the patient's body.

### CROSS-REFERENCE TO RELATED APPLICATIONS

The application claims priority to Chinese patent application No. 202410106574.0, filed on Jan. 25, 2024, the entire contents of which are incorporated herein by reference.

### TECHNICAL FIELD

The present invention relates to the field of ventilator technology, and particularly to a multi-functional breathing tube for humidification without water.

### BACKGROUND

Generally speaking, the ventilator is a kind of instrument commonly used in hospitals and families to stabilize the patient's breathing and treat the patient. The patient will wear a nose and mouth mask, and then connect with the nose and mouth mask through the ventilator's gas pipe, and communicate with the patient through the gas pipe to deliver oxygen. Respiratory nebulization therapy is an important means for the treatment of respiratory diseases. During treatment, the nebulizer disperses water and drugs into tiny droplets suspended in the gas and enters the respiratory tract and lungs through the patient's breathing. The nebulizer is an important part of the ventilator, which can improve the user's comfort. From a long-term perspective, it can play a certain role in moistening and protecting the nasal mucosa and upper respiratory tract of patients, avoiding the occurrence of some related phenomena such as dry nose, dry upper respiratory tract and dry mouth under the treatment of breathing for a long time.

However, the existing medical atomizer has a large volume, is very inconvenient to use and carry, and the atomizing gas path is long, and the atomizing liquid is easy to condense in the spray tube, which affects the drug utilization rate and atomization effect. The traditional humidifier is generally designed on the ventilator, which is large in volume and needs to be humidified with water. When used at night, the host is easy to backfill and dry burn, which is easy to cause fire and other safety accidents.

### SUMMARY

The invention provides a multi-functional breathing tube for humidification without water, which overcomes the problem of low utilization rate of hose space in the prior art by improving the humidification mode and increasing the functions of atomization and sputum discharge.

To solve the above problems, the technical scheme provided by the invention is as follows:

The embodiment of the invention provides a multi-functional breathing tube for humidification without water. The multi-functional breathing tube for humidification without water includes a breathing mask (1), the breathing mask (1) is connected with a sputum ejector (2), the sputum ejector (2) is connected with an anhydrous humidifier atomizer (3), and the anhydrous humidifier atomizer (3) is connected with a medicated bin (4). The medicated bin (4) is connected with a breathing hose (5), the breathing hose (5) is connected with an air source device, and the gas output by the air source device is oxygen.

According to an optional embodiment of the invention, the sputum ejector (2) has an inner cavity structure (203) and an outer cavity structure (204), the inner cavity structure (203) of the sputum ejector (2) is communicated with the breathing mask (1), and the outer cavity structure (204) of the sputum ejector (2) is communicated with the anhydrous humidifier atomizer (3); The outer cavity structure (204) of the sputum ejector (2) is communicated with the end respiratory mouth of the inner cavity structure (203) of the sputum ejector (2); A one-way valve (201) is arranged on the end face of the sputum ejector (2) facing the breathing mask (1), and a pendulum ball (202) is arranged in the inner cavity structure (203) of the sputum ejector (2). When the patient exhales, the gas pushes the pendulum ball (202) to deflate and swing, causing 0-30 Hz vibration, resonating with the sputum in the human body, loosening the sputum and facilitating sputum ejection; When the patient exhales the positive pressure of the breathing cavity, the sputum is pressed out from the bottom of the sputum ejector (2), and when the patient is lying down the pendulum ball (202) tilts due to gravity, the end of the breathing mouth of the inner cavity structure of the sputum ejector (2) is opened.

According to an optional embodiment of the invention, the anhydrous humidifier atomizer (3) comprises a micro-conical hole plate (304), a piezoelectric plate (301) connected with the micro-conical hole plate (304), a battery compartment (302) connected with the piezoelectric plate (301), and a metal substrate (303) located on both sides of the battery compartment (302); The anhydrous humidifier atomizer (3) has an intermediate intake channel (306) and an edge intake channel (305) arranged around the intermediate intake channel (306); The intermediate intake channel (306) and the edge intake channel (305) of the intermediate intake channel (306) are communicated by the outer cavity structure (204) of the phlegm ejector (2).

According to an optional embodiment of the invention, The medicated bin (4) comprises a dosing body, the dosing body is provided with an atomizing capsule (403), the side wall of the atomizing capsule (403) is a rubber wall (404), and the outlet wall of the atomizing capsule (403) is a polymer wall surface (402). The polymer wall surface (402) is connected with an absorbent sheet (401).

The medicated bin (4) also comprises a main intake channel (405), a main intake channel (405) is located outside the atomizing capsule (403), one end of the main intake channel (405) is communicated with the breathing hose (5), the other end of the main intake channel (405) is communicated with the edge intake channel (305) of the anhydrous humidifier atomizer (3), The gas channel of the outlet air wall of the atomizing capsule (403) is communicated with the intermediate intake channel (306) of the anhydrous humidifier atomizer (3).

According to an optional embodiment of the invention, the piezoelectric plate (301) is a microporous piezoelectric ceramic plate.

Beneficial effects: (1) the phlegm ejector is connected with the anhydrous humidifier atomizer, and the pendulum ball in the phlegm ejector resonates with the sputum when the patient exhales deeply, and assists the sputum ejector outside the patient; The breathing mask is connected with the anhydrous humidifier atomizer, which effectively reduces the volume of the ventilator, increases the function of the nebulizer, and is convenient to carry; By breathing to change the size of the atomizing capsule of the medicated bin to feed the liquid, more convenient to carry. (2) The invention makes the breathing gas more humid, protects the respiratory tract of the patient, alleviates the pain of some patients with respiratory diseases, and brings comfortable experience to the patient through the microporous piezoelectric ceramic plate. (3) The invention uses human respiration to design a detachable and mounting pharmaceutical storage bin. The change of respiratory pressure causes the dosing bin to shrink, and then releases the pharmaceutical liquid, which enters the human respiratory system with respiration, enhances the health care function, increases or decreases the detachable and mounting pharmaceutical storage bin, and has good curative effect on rhinitis, cough, pharyngitis, pulmonary infection, etc. (4) The invention drives airway air to vibrate by blowing and swinging the ball, resonates with the inner wall cilia deep in the airway, loosens the phlegm stuck in the airway, and thus leaves the airway surface.

### BRIEF DESCRIPTION OF DRAWINGS

In order to more clearly illustrate the technical scheme in the embodiment or prior art, the following is a brief introduction to the drawings required to be used in the description of the embodiment or prior art. It is obvious that the drawings in the description below are only embodiments of the invention. Other drawings can also be obtained from these drawings.

FIG. 1 is a structural diagram of a multi-functional breathing tube for humidification without water provided for the embodiment of the present application.

FIG. 2 is a schematic diagram of the decomposition structure of a multi-functional breathing tube for humidification without water provided for the embodiment of this application.

FIG. 3 and FIG. 4 provide a structural diagram of a sputum ejector for the embodiment of this application.

FIG. 5 is a structural diagram of an anhydrous humidifier atomizer provided for the embodiment of this application.

FIG. 6 is a structural diagram of a micro-conical hole plate of an anhydrous humidifier atomizer provided for the embodiment of this application.

FIG. 7 is a structural diagram of a medicated bin provided for the embodiment of this application.

FIG. 8 is a schematic diagram of normal airflow propagation of a multi-functional breathing tube for humidification without water provided for the embodiment of this application.

FIG. 9 is a schematic diagram of inspiratory air propagation of a multi-functional breathing tube for humidification without water provided for the embodiment of this application.

FIG. 10 is a schematic diagram of expiratory air propagation of a multi-functional breathing tube for humidification without water provided for the embodiment of the present application.

### DETAILED DESCRIPTION OF THE EMBODIMENTS

The technical scheme in the embodiment of this application will be clearly and completely described in combination with the drawings attached to the embodiment of this application. It is clear that the embodiment described are only a portion of the embodiment of this application and not the entirety of the embodiment. Based on the embodiment in this application, all other embodiments obtained by a person skilled in the field without performing creative labor fall within the scope of protection in this application.

Aiming at the problem that the humidifier in the prior art is designed on the ventilator with large volume and needs to be humidified with water, and the main engine is easy to backfill and dry burn when used at night, which is easy to cause fire and other safety accidents, the invention provides a multi-functional breathing tube for humidification without water, which can solve the above technical problems.

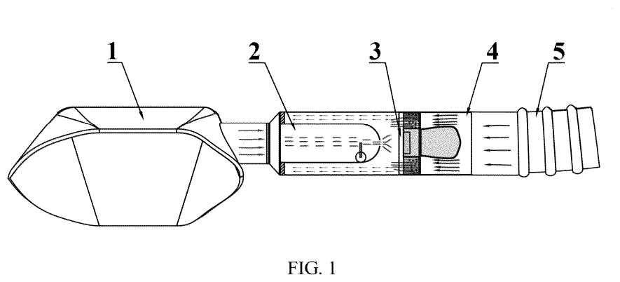

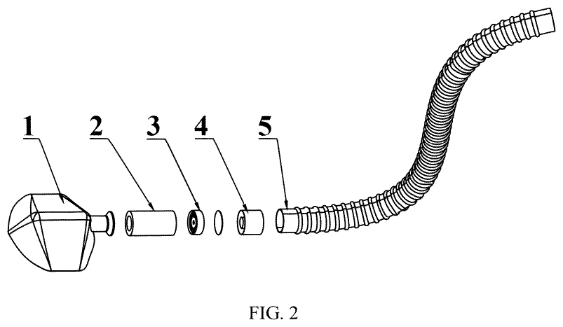

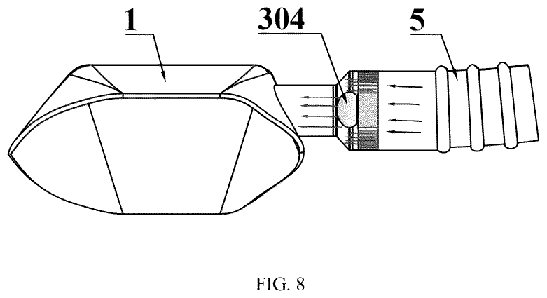

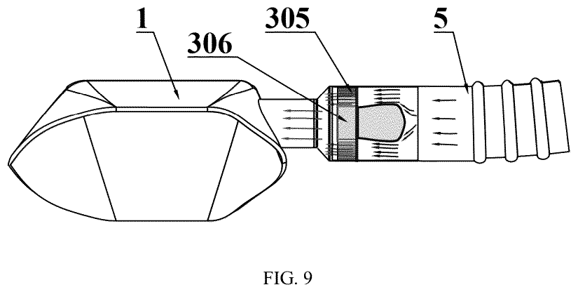

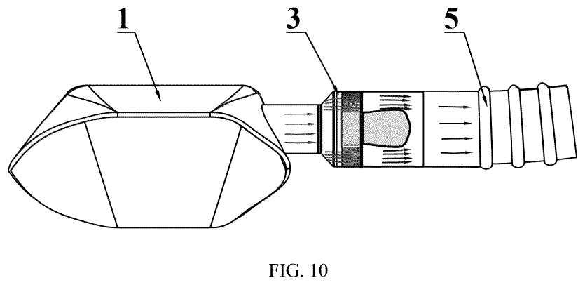

As shown in FIG. 1, FIG. 2, FIG. 8, FIG. 9 and FIG. 10, The multi-functional breathing tube for humidification without water includes a breathing mask 1, the breathing mask 1 is connected with a sputum ejector 2, the sputum ejector 2 is connected with an anhydrous humidifier atomizer 3, and the anhydrous humidifier atomizer 3 is connected with a medicated bin 4, the medicated bin 4 is used to place atomizing agents or other agents. the medicated bin 4 is connected with a breathing hose 5, the breathing hose 5 is connected with the air source device, and the gas output by the air source device is oxygen. Compared with the prior art, the invention connects the sputum ejector 2, the anhydrous humidifier atomizer 3 and the medicated bin 4 to the pipeline, effectively reduces the volume of the ventilator, increases the function of the nebulizer, and delivers the liquid by breathing and changing the volume size of an atomizing capsule 403 of the medicated bin 4, which is more convenient to carry. The sputum ejector 2 is connected with the anhydrous humidifier atomizer 3, resonates with the sputum during deep exhalation, and assists the exhalation.

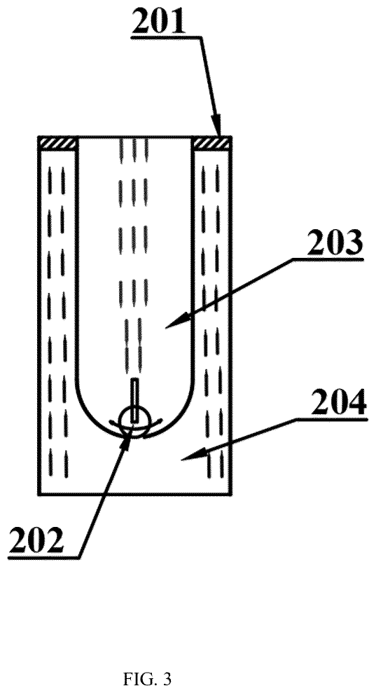

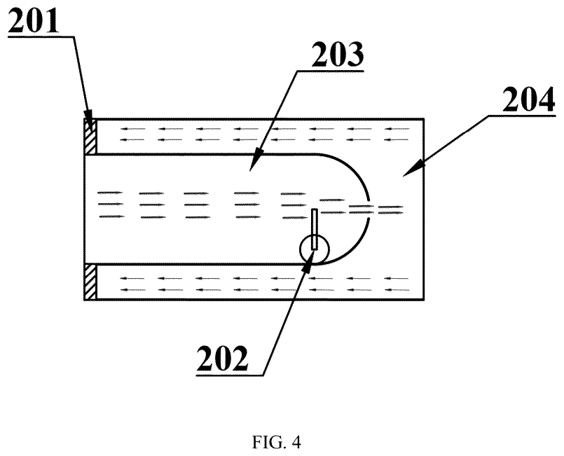

As shown in FIG. 3 and FIG. 4, the sputum ejector 2 has an inner cavity structure 203 and an outer cavity structure 204, the inner cavity structure 203 of the sputum ejector 2 is communicated with breathing mask 1, and the outer cavity structure 204 of the sputum ejector 2 is communicated with the anhydrous humidifier atomizer 3. The outer cavity structure 204 of the sputum ejector 2 is connected with the end respiratory mouth of inner cavity structure 203 of the sputum ejector 2; A one-way valve 201 is arranged on one end face of the sputum ejector 2 facing the breathing mask 1, and a pendulum ball 202 is arranged in the inner cavity structure 203 of the sputum ejector 2. When the patient exhales, the gas pushes the pendulum ball 202 to deflate and swing, causing 0-30 Hz vibration, resonating with the sputum in the human body, loosening the sputum and facilitating sputum drainage. When the patient exhales, the positive pressure of the breathing cavity will press the sputum from the bottom of the sputum ejector 2. When the patient is lying down, the pendulum ball will tilt due to gravity, and the end of the inner cavity structure of the sputum ejector 2 will open.

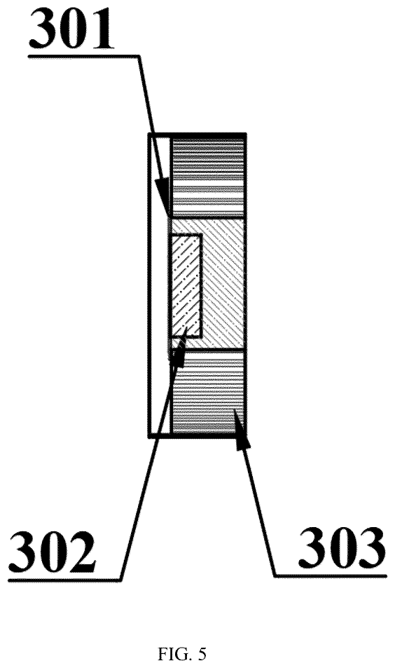

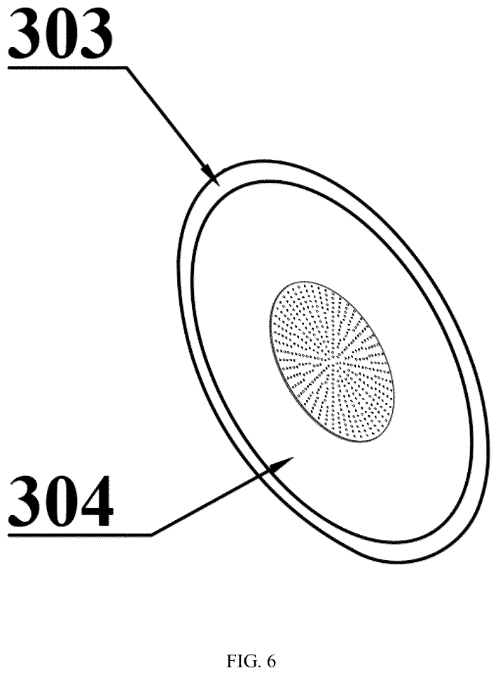

As shown in FIGS. 5 and 6, the anhydrous humidifier atomizer 3 comprises a micro-cone hole plate 304, a piezoelectric plate 301 connected with a micro-cone hole plate 304, a battery compartment 302 connected with a piezoelectric plate 301, and a metal substrate 303 located on both sides of the battery compartment 302. The anhydrous humidifier atomizer 3 has an intermediate intake channel 306 and an edge intake channel 305 arranged around an intermediate intake channel 306, refer to FIG. 9. Both the intermediate intake channel 306 and the edge intake channel 305 of the anhydrous humidifier atomizer 3 are connected by the outer cavity structure 204 of the sputum ejector 2. Piezoelectric plate 301 is a microporous piezoelectric ceramic plate.

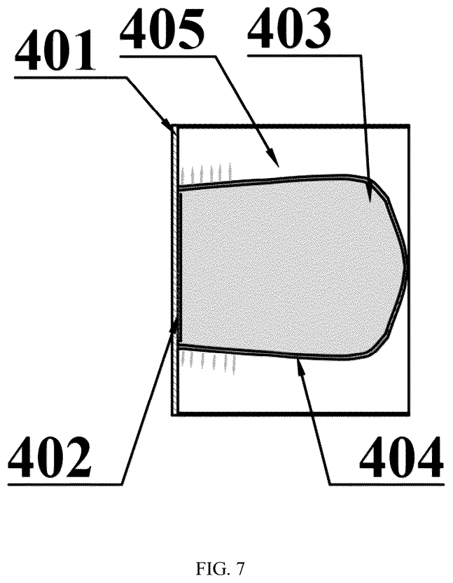

As shown in FIG. 7, The medicated bin 4 includes the dosing body. The dosing body is provided with an atomizing capsule 403. The side wall of the atomizing capsule 403 is rubber wall 404, the outlet wall of atomizing capsule 403 is a polymer wall surface 402, and a polymer wall surface 402 is connected with an absorbent sheet 401.

The medicated bin 4 also includes a main intake channel 405, which is located outside the atomizing capsule 403, one end of the main intake channel 405 is communicated with the breathing hose 5, the other end of the main intake channel 405 is communicated with the edge intake channel 305 of the anhydrous humidifier atomizer 3, The gas channel of the outlet wall of the atomizing capsule 403 is communicated with the intermediate intake channel 306 of the anhydrous humidifier atomizer 3.

FIG. 8 is a schematic diagram of normal airflow propagation of a multi-functional breathing tube for humidification without water provided for the embodiment of this application. Oxygen is output from the air source device, oxygen goes through the breathing hose 5, oxygen goes through the main intake channel 405 of the medicated bin 4, oxygen goes through the edge intake channel 305 on both sides of the anhydrous humidifier atomizer 3, oxygen goes through the outer cavity structure 204 of the sputum ejector 2, through the outer cavity structure of the sputum ejector 2, through the breathing mask 1, and input to the patient's mouth. The atomizing capsule 403 of the medicated bin 4 is deployed towards the sputum ejector 2 under the action of air flow.

The patient in this embodiment needs to be seated while excreting sputum, the pipe is in a vertical direction, the body is under negative pressure during inhalation, and the gas is inhaled through a one-way check valve. During exhalation, the gas pushes the ball to deflate and swing, causing 0-30 Hz vibration, resonating with the sputum in the body, loosening the sputum and facilitating sputum expulsion. Positive pressure in the breathing cavity as you exhale pushes sputum out of the bottom. When the patient is lying down, the pendulum tilts due to gravity and the breathing mouth opens.

FIG. 9 is a schematic diagram of inspiratory air propagation of a multi-functional breathing tube for humidification without water provided for the embodiment of this application. When inhaling, the atomizing capsule 403 of the medicated bin 4 is deployed towards the breathing hose 5 under the action of the air flow. The piezoelectric plate 301 atomizes the absorbed water vapor, humidifies and heats the gas of the respirator during inhalation; The fan flows into the medicated bin 4 in the same direction as the gas inhaled by the human body, and the gas flowing through the outside of the medicated bin 4 has a faster flow rate. According to the Bernoulli principle, at this time, the pressure inside the pipeline is low, and the medicated bin 4 expands outward, and the liquid inside the expanded medicated bin is extruded and transferred to the piezoelectric plate for atomization through the capillary action of the absorbent material. By combining the polymer material with the rubber and plastic material, the agent will flow out of the medicine bin when it expands.

FIG. 10 is a schematic diagram of expiratory air propagation of a multi-functional breathing tube for humidification without water provided for the embodiment of the present application. When exhaling, the exhaled gas passes through the breathing mask 1, flows to the inner cavity structure 203 of the sputum ejector 2, flows to the outer cavity structure 204 of the sputum ejector 2, passes through the outer cavity of the medicated bin 4, and passes through the breathing hose 5. The atomizing capsule 403 of the medicated bin 4 is deployed towards the breathing hose 5 under the action of the exhaled gas. The exhalation porous structure captures water vapor and heat, at this time, the fan pushes the gas into the direction opposite to the direction of the human body exhaled gas, the flow rate is low, and the dosing bin of the rubber and plastic material remains unchanged.

In summary, although the invention has been disclosed as above in preferred embodiment, the said preferred embodiment is not intended to limit the invention, and persons of ordinary skill in the art may make various changes and refinements within the spirit and scope of the invention, so that the scope of protection of the invention shall be defined by the claims.

What is claimed is:

1. A multi-functional breathing tube for humidification without water, it includes a breathing mask (1), the breathing mask (1) is connected with a sputum ejector (2), the sputum ejector (2) is connected with an anhydrous humidifier atomizer (3), and the anhydrous humidifier atomizer (3) is connected with a medicated bin (4); The medicated bin (4) is connected with a breathing hose (5), the breathing hose (5) is connected with an air source device, and the gas output by the air source device is oxygen.

2. As the multi-functional breathing tube for humidification without water described in claim 1, wherein, the sputum ejector (2) has an inner cavity structure (203) and an outer cavity structure (204), the inner cavity structure (203) of the sputum ejector (2) is communicated with the breathing mask (1), and the outer cavity structure (204) of the sputum ejector (2) is communicated with the anhydrous humidifier atomizer (3); The outer cavity structure (204) of the sputum ejector (2) is communicated with the end respiratory mouth of the inner cavity structure (203) of the sputum ejector (2); A one-way valve (201) is arranged on the end face of the sputum ejector (2) facing the breathing mask (1), and a pendulum ball (202) is arranged in the inner cavity structure (203) of the sputum ejector (2); When the patient exhales, the gas pushes the pendulum ball (202) to deflate and swing, causing 0-30 Hz vibration, resonating with the sputum in the human body, loosening the sputum and facilitating sputum ejection; When the patient exhales the positive pressure of the breathing cavity, the sputum is pressed out from the bottom of the sputum ejector (2), and when the patient is lying down the pendulum ball (202) tilts due to gravity, the end of the breathing mouth of the inner cavity structure of the sputum ejector (2) is opened.

3. As the multi-functional breathing tube for humidification without water described in claim 2, wherein, the anhydrous humidifier atomizer (3) comprises a micro-conical hole plate (304), a piezoelectric plate (301) connected with the micro-conical hole plate (304), a battery compartment (302) connected with the piezoelectric plate (301), and a metal substrate (303) located on both sides of the battery compartment (302); The anhydrous humidifier atomizer (3) has an intermediate intake channel (306) and an edge intake channel (305) arranged around the intermediate intake channel (306); The intermediate intake channel (306) and the edge intake channel (305) of the intermediate intake channel (306) are communicated by the outer cavity structure (204) of the phlegm ejector (2).

4. As the multi-functional breathing tube for humidification without water described in claim 3, wherein, the medicated bin (4) comprises a dosing body, the dosing body is provided with an atomizing capsule (403), the side wall of the atomizing capsule (403) is a rubber wall (404), and the outlet wall of the atomizing capsule (403) is a polymer wall surface (402); The polymer wall surface (402) is connected with an absorbent sheet (401);
The medicated bin (4) also comprises a main intake channel (405), a main intake channel (405) is located outside the atomizing capsule (403), one end of the main intake channel (405) is communicated with the breathing hose (5), the other end of the main intake channel (405) is communicated with the edge intake channel (305) of the anhydrous humidifier atomizer (3), The gas channel of the outlet air wall of the atomizing capsule (403) is communicated with the intermediate intake channel (306) of the anhydrous humidifier atomizer (3).

5. As the multi-functional breathing tube for humidification without water described in claim 3, wherein, the piezoelectric plate (301) is a microporous piezoelectric ceramic plate.
